(19) United States
(12) Patent Application Publication
(10) Pub. No.: US 2002/0197325 A1
(43) Pub. Date: Dec. 26, 2002

(54) Microparticles

(75) Inventor: Nicholas D. Osborne, Colwick (GB)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Quadrant Healthcare (UK) Limited

(21) Appl. No.: 09/797,181

(22) Filed: Mar. 2, 2001

Related U.S. Application Data
(63) Continuation of application No. PCT/GB99/02930, filed on Sep. 3, 1999.

(30) Foreign Application Priority Data
Sep. 3, 1998 (GB) 9819272.7

(51) Int. Cl. A61K 9/14; B29B 9/00

(52) U.S. Cl. 424/489; 264/12

(57) ABSTRACT

Microparticles comprising or consisting of a therapeutic agent have a particle density of at least 80% of the solid agent and a shape factor of 1 to 5. The microparticles may be produced by spray drying and may be used in needleless injection.

### FIELD OF THE INVENTION

This invention relates to microparticles, methods for their formation and their therapeutic use, especially for the delivery of active agents through the skin using needleless injection systems.

### BACKGROUND OF THE INVENTION

Needleless injectors use compressed gas to accelerate particles to a velocity at which they are capable of penetrating skin and mucosal barriers; such devices are described in WO-A-94/24263. A requirement is that the particles have mechanical strength, and it is advantageous to have a high density. It is also beneficial to use particles having uniform shape, preferably spherical, and a controlled size distribution; these factors affect the aerodynamic behaviour and the penetration of the particles, and hence the efficacy of the delivery of the active agent. Useful particles typically have a size in the range of 10-500 μm.

The production of solid or dense microparticles can be achieved by milling, e.g. micronisation of larger particles, crystallisation, precipitation or another solution-based microparticle generation technique. However, these techniques typically do not produce spherical microparticles.

A technique which does not normally produce solid microparticles is spray-drying, where often low density particles and agglomerates are formed. A major industry where high density products are important is the dairy industry where skimmed milk powders are produced (Spray Drying Handbook, K. Masters, 5th Edition, 1991, Longman Scientific and Technical, pages 330-336). In this section, products produced by conventional spray-drying are shown on photomicrographs where it is stated that they contain “vacuoles”, are of “low density”, are thin-walled, “cannot withstand mechanical handling and are readily fragmented”, and are obtained together with high and low amounts of occluded air. Some increase in density is described by using a more complicated, two-stage spray-drying process which produces contorted and shrivelled particles. Charlesworth and Marshall, J. Appl. Chem. Eng., 6 No. 1, 9 (1960), describes the morphology of particles produced from spray-drying where all the particles are porous, sponge-like or contain occluded air as a result of collapsing, blistering, bubbling or expansion. Examples of processes in which the inclusion of air is optimised in a spray-drying process are described in WO-A-92/18164, WO-A-96/09814 and WO-A-96/18388.

### SUMMARY OF THE INVENTION

Surprisingly, it has been found that dense microspheres of solid or semi-solid form can be produced from materials using carefully controlled spray-drying conditions. These microspheres are particularly suitable for use in needleless injection systems due to their density and sphericity. More particularly, the relative particle density may be at least 80%, often at least 90% and even 100% of the solid material. The sphericity is usually such that the shape factor is 1 to 5.

Accordingly, a first aspect of the invention involves microparticles comprising or consisting of a therapeutic agent, having a relative particle density of at least 80% of the solid agent, and a shape factor of 1 to 5.

In a second aspect, the invention provides the use of a therapeutic agent for the manufacture of a medicament in the form of microparticles of the invention, for administration by needleless injection.

A third aspect of the invention is a needleless syringe comprising the microparticles of the invention.

In a fourth aspect, the invention is a method of therapeutic treatment which comprises the transdermal, transmucosal or subcutaneous delivery of microparticles of the invention using a needleless syringe.

According to the invention in a fifth aspect, there is provided a method of producing the microparticles of the invention which comprises spray-drying a solution or suspension comprising the therapeutic agent.

### DESCRIPTION OF THE INVENTION

Aspects of the present invention are illustrated, by way of example only, in the accompanying drawings, in which:

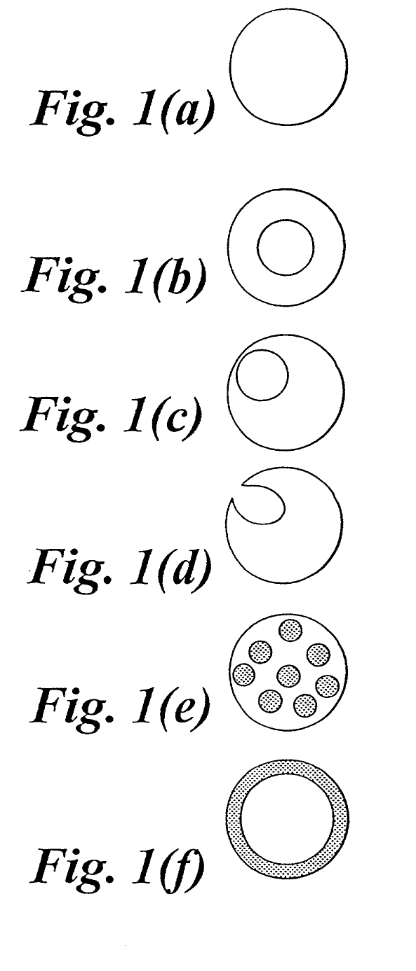

FIG. 1 shows, schematically, microparticles of the invention;

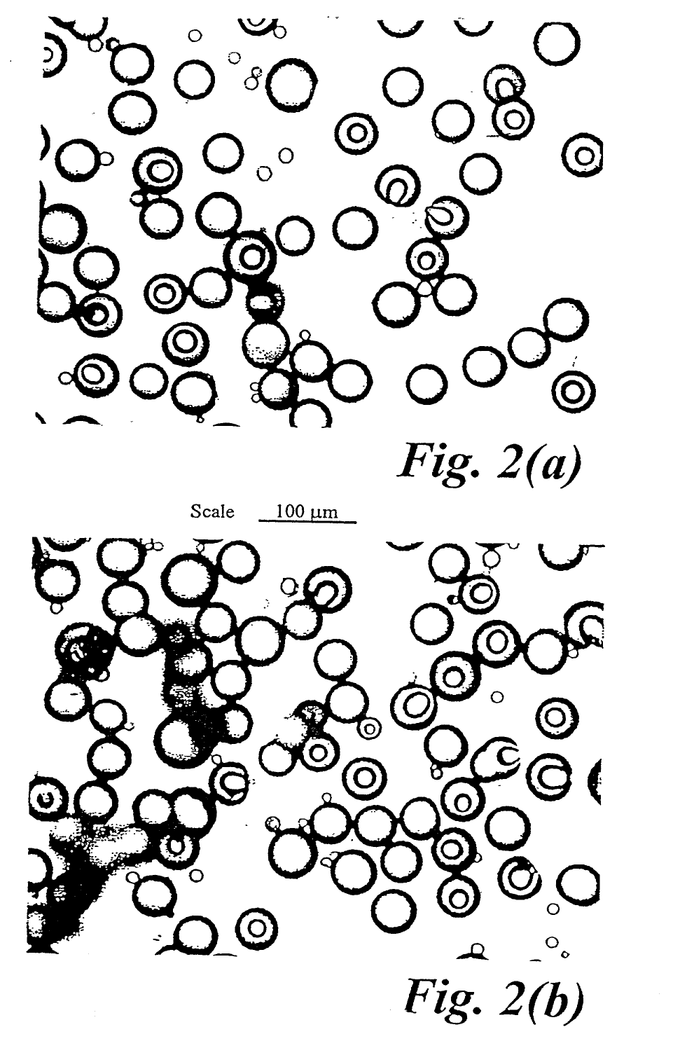

FIGS. 2A and 2B are photomicrographs of the product of Example 1;

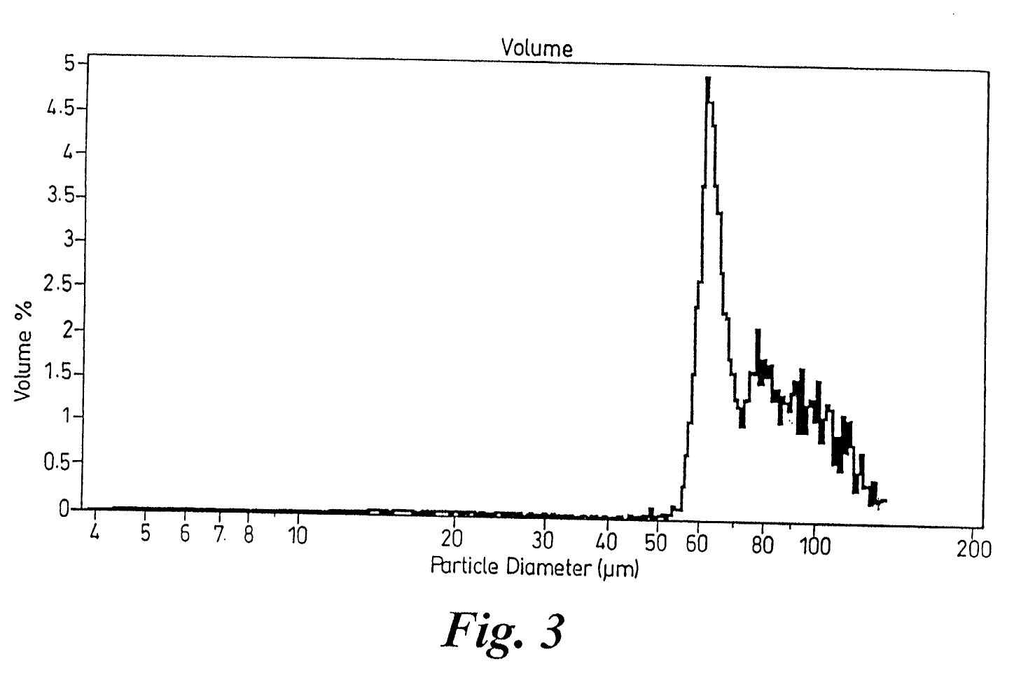

FIG. 3 shows the particle size distribution for the product of Example 1;

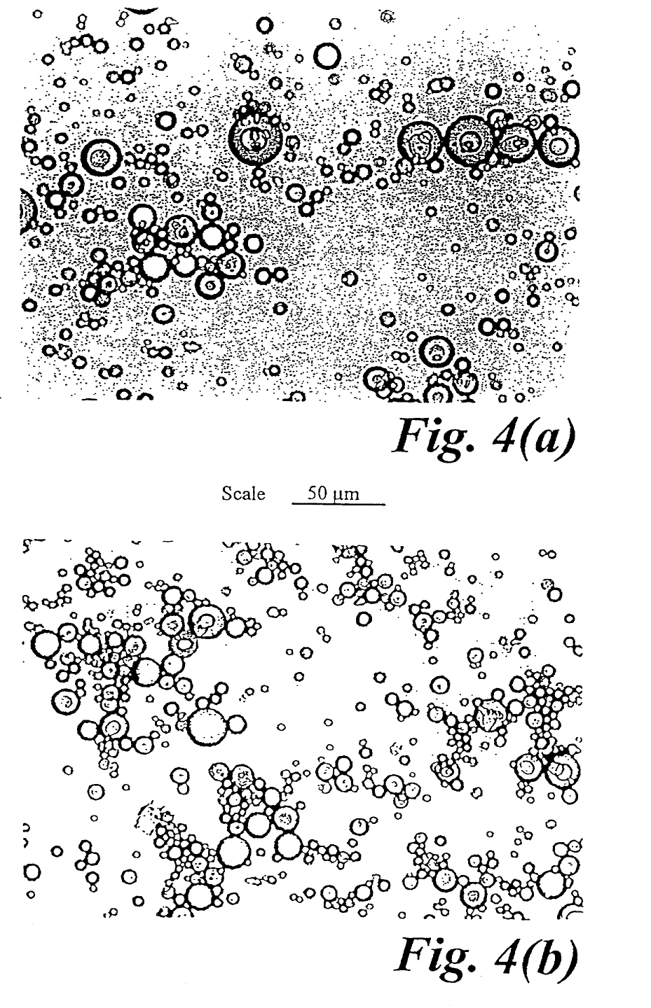

FIGS. 4A and 4B are photomicrographs of the product of Example 2;

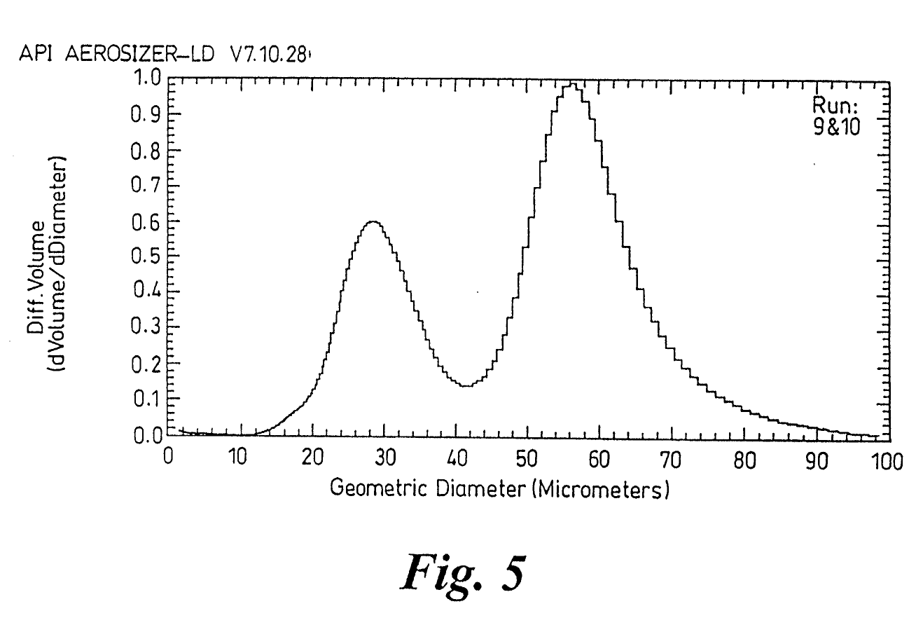

FIG. 5 shows the particle size distribution for the NT2TRE1 product of Example 3;

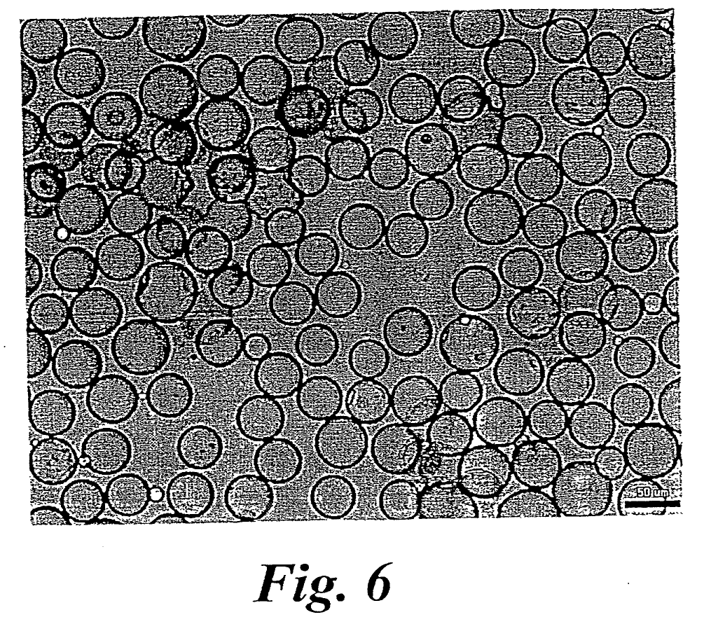

FIG. 6 is an optical micrograph of the NT2TRE3 product of Example 5 retained after sieving;

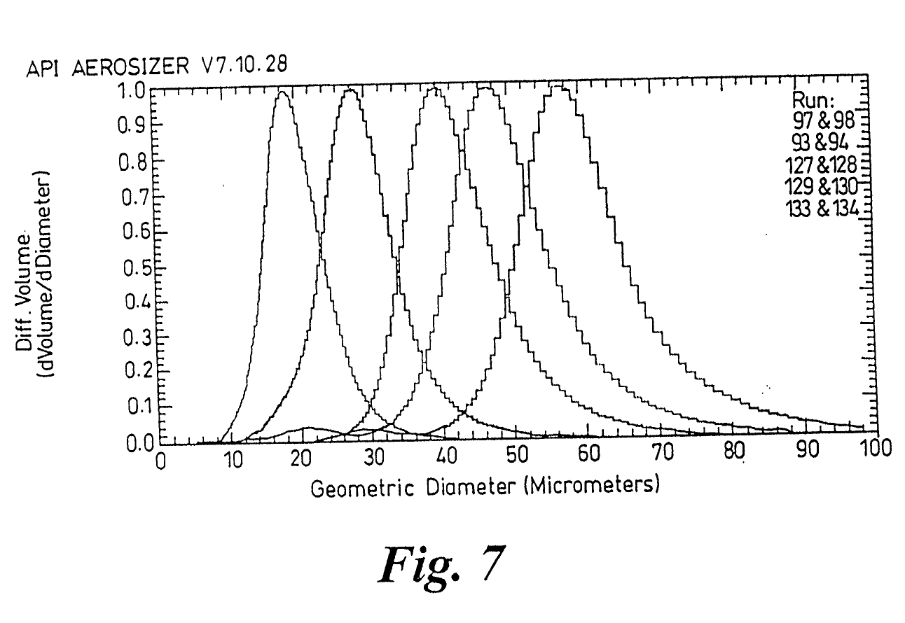

FIG. 7 shows the size distribution of the sieved products of Example 5;

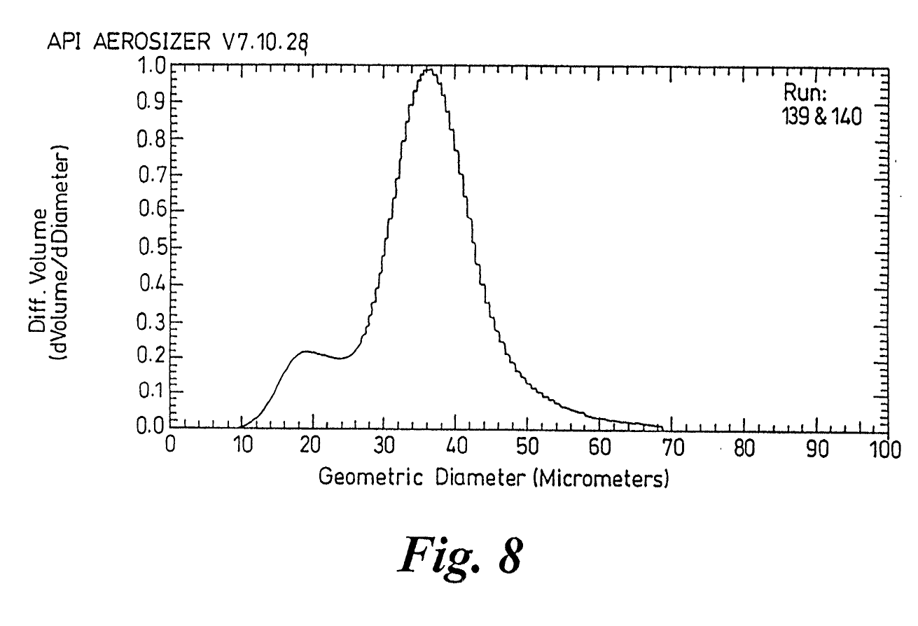

FIG. 8 shows the particle size distribution for the product of Example 7.

The solid or semi-solid microspheres of the invention produced, also referred to herein as microparticles, can be in a variety of forms, examples of which are shown in FIG. 1. In addition to (a) solid spheres, semi-solid spheres can be formed; these are where (b) a small air pocket is occluded in the centre, (c) an occlusion is off centre, or (d) an occlusion has broken out of the microsphere.

Many references, including the Spray Drying Handbook, commonly refer to bulk densities, calculated from the volume which a given mass occupies. In connection with this invention, the particle density is more important; this is based on the volume of the particle including any closed inclusions but not any open structures. Hence, the forms shown in FIG. 1(a) and (d) have identical particle densities but (b) and (c) have lower (and identical) particle densities.

A solid microsphere has a particle density identical to the material it is formed from and has a relative particle density of 100%. If small air inclusions are present, the relative particle density is less than 100%. The average particle density can be measured by liquid or gas pycnometry or calculated for individual microspheres using measurements made by optical microscopy. The density of the therapeutic agent is measured at 25° C. From these measurements the-microspheres of this invention have relative particle densities of at least 80% and preferably more than 90%, 95%, 99% or 100% of the original material. For application to needleless injection systems, high relative particle densities are required to give mechanical strength and the given relative densities are suitable. In particular, the microspheres can meet the requirements set out, for needleless injection, in WO-A-94/24263, the contents of which are incorporated herein by reference.

Active materials, which the microparticles of the invention may comprise or consist of and which may be delivered by needleless injection, are therapeutic agents including pharmacologically active substances, which are generally solids. Therapeutic agents which may be delivered include, for example, proteins, peptides, nucleic acids and small organic molecules, for example local anesthetics (such as cocaine, procaine and lidocaine), hypnotics and sedatives (such as barbiturates, benzodiazepines and chloral derivatives), psychiatric agents (such as phenothiazines, tricyclic antidepressants and monoamine oxidase inhibitors), anti-epilepsy compounds (such as hydantoins), L-dopa, opium-based alkaloids, analgesics, anti-inflammatories, allopurinol, cancer chemotherapeutic agents, anticholinesterases, sympathomimetics (such as epinephrine, salbutamol and ephedrine), antimuscarinics (such as atropine), α-adrenergic blocking agents (such as phentolamine), β-adrenergic blocking agents (such as propranolol), ganglionic stimulating and blocking agents (such as nicotine), neuromuscular blocking agents, autacoids (such as anti-histamines and 5-HT antagonists), prostaglandins, plasma kinins (such as bradykinin), cardiovascular drugs (such as digitalis), antiarrhythmic drugs, antihypertensives, vasodilators (such as amyl nitrate and nitroglycerin) diuretics, oxytocin, antibiotics, anthelminthics, fungicides, antiviral compounds (such as acyclovir), anti-trypanosomals, anticoagulants, sex hormones (for example for HRT or contraception), insulin, alprostidil, blood-clotting factors, calcitonin, growth hormones, vaccines, constructs for gene therapy and steroids. The recipient may be a human or any other vertebrate, preferably a mammal, bird or fish for example a cow, sheep, horse, pig, chicken, turkey, dog, cat or salmon, or a plant, especially for DNA transformation of the plant. For example, DNA is generally presented as a plasmid and may, for example, be the DNA encoding an anti-Chlamydia antigen disclosed in Vanrompay et al (1999) Vaccine 17, 2628-2635. Vaccines may take the form of proteins or other polypeptides or oligopeptides, or DNA encoding an antigen, for example DNA encoding an HIV or hepatitis B antigen. The microspheres may be formed from the active material alone, or they may contain one or more excipients or stabilisers including proteins, sugars, antiseptics, preservatives and buffers. Carbohydrates and other glass-forming substances may be employed as stabilisers or excipients. Preferably, the excipients are parenterally acceptable. If an excipient is present, the active compound may be uniformly distributed or be in the form of smaller particles entrapped in a matrix, as shown in FIG. 1(e). Suitable carbohydrates that may be used are as disclosed in WO 96/03978. Hydrophobically derivatised carbohydrates, as disclosed in WO 96/03978, may be used to provide a controlled release form of the particles.

A further embodiment of this invention is the use of excipients or additives with higher density than the active substance or excipient to form even higher density microspheres.

Microspheres of this invention are typically of defined sizes with 95% or more of the particles (by weight) having a size in the range of 10-500 μm, preferably 20-200 μm, and most preferably 30-100 μm. The modal distribution may be centred around 10 μm bands, i.e. 30, 40, 50, 60, 70, 80, 90 and 100 μm. Preferably, in a monomodal sample, 80% of the particles by weight are within a size range of 10 μm for the particles of a smaller size to a size range of 25 μm for the particles having a larger size (the range increasing with the size of the particles), more preferably, 90% of the particles are within a size range of 15 μm (for the smaller particles) to 30 μm (for the larger particles).

The microspheres of the invention may be formed with a bimodal distribution of particles sizes. Typically, when a rotary atomiser is used, at least 60%, such as more than 75%, by weight of the particles have particle sizes distributed about one modal size and the remaining particles have particle sizes distributed about a smaller modal size. Where a monomodal particle size distribution is required, the smaller particles may be separated from the larger particles by routine techniques, such as sieving, for example. Microparticles having other distributions of particle sizes can also be obtained in the invention.

The sphericity of the particles is also important and is defined as the shape factor which is the true surface area divided by the equivalent spherical area for the particle volume. The particle surface area can be found by using the standard technique of nitrogen adsorption with subsequent BET analysis. The microspheres of this invention typically have a shape factor of 1 to 5, preferably 1 to 2. Alternative techniques for assessing shape can be found from optical microscopy aided by image analysis to measure circularity and elongation which give similar values to the shape factor.

The microspheres are generally made by spray-drying a solution or suspension of the material. Suitable solvents for most pharmacologically active substances are known. Water is the preferred solvent. The concentration of the material can be varied in order to arrive at the desired solid microparticles but 0.1 to 70% solutions, preferably 10-30% solutions, can be suitable. If the microparticles do not consist of the active material, from the carriers mentioned above, such as a relatively inert protein (such as human serum albumin, preferably produced by rDNA techniques) or sugar (such as trehalose), may be used. Water is again the preferred solvent.

The concentration of active ingredient in the sprayed solution or suspension, and the ratio of the active ingredient to the carrier material (if present) will generally be governed by the amount of the particles to be delivered by the injector and the dose of active ingredient desired.

A conventional spray dryer may be used, e.g. a pilot scale spray dryer atomising the liquid feed solution or suspension by either a pressure nozzle or two fluid atomisation, although rotary atomisers are preferred. The formation of suitable solid or semi-solid microspheres may be dependent on the use of low outlet temperatures in the drying process, for certain therapeutic agents or mixtures of therapeutic agents and excipients. Suitable outlet temperatures can be readily determined by the skilled person for any given therapeutic agent or mixture of therapeutic agent and excipient. The inlet temperature is set to give the required outlet temperature based on the type of atomisation used and other variables such as drying airflow rate; it may be, for example, 50-270° C. The particle size is controlled by standard parameters for the atomiser used at a given feed concentration.

The microspheres may be farther dried, following their formation by spray-drying, to remove residual water or solvent by the use of heat and/or vacuum. Suitable drying techniques for this farther drying step include, for example, fluidised bed drying. The use of a fluidised bed for this further drying step has the advantage that, when the microspheres have a bimodal particle distribution, the small particles may be separated from the larger particles by elutriation. The formation of crystals should be avoided.

The microspheres may also be coated using standard techniques, e.g. fluid bed coating, to add a further layer or layers to alter the release profile or protect the active compound, as shown in FIG. 1 (e) . The particle size distribution produced may also be modified to select a particular size range using sieving or other commercial classification techniques to further define particle distribution.

The microspheres may be sterilised, depending on their application. A sterile product can be achieved through either aseptic manufacturing or terminal sterilisation, e.g. gamma irradiation.

Examples of needleless syringes which may be used to deliver the microparticles of the invention and component parts thereof are shown in WO 94/24263 (issued as U.S. Pat. No. 5,899,880 and U.S. Pat. No. 5,630,796, which are incorporated herein by reference).

The syringe is typically some 18 cm long, although it may be smaller or larger than this, and is arranged to be held in the palm of the hand with the thumb overlying the upper end.

In order to carry out an injection, the wider end of the spacer shroud of the device is pressed against a patient's skin. The gas released from a reservoir into a chamber eventually creates in the chamber a pressure sufficient to burst two diaphragms and allow the gas to travel through a nozzle, with the particles entrained thereby, into the patient's skin.

The chamber may be prefilled with gas, such as helium, at a superatmospheric pressure of, say, 2-4 bar, but possibly even as high as 10 bar. The particles of the invention are thus entrained in (ie suspended in) a gas such as helium at the moment of delivery.

The following Examples further illustrate the invention.

### EXAMPLE 1

100 ml of diafiltered aqueous 20% w/v (weight by volume) HSA solution (as a model for a pharmacologically active protein, or as the carrier for a pharmacologically active compound) was spray dried on a Niro Mobile Minor spray dryer using a NT2 rotary atomiser (Newland Design, Lancaster) at the following conditions:

| | |
|---|---|
| Inlet Temperature | 245° C. |
| Outlet Temperature | 35° C. |
| Feed Rate | 10 g/min |
| Rotational Speed | 30,000 rpm |

The outlet temperature is low as additional air was supplied to guide the droplets into the drying chamber.

A water soluble product was obtained of which photomicrographs can be found in FIG. 2. These show that over 65% of the microspheres were solid with a uniform size of around 50 μm. The similarly sized microspheres containing small amounts of air had thick walls and calculated densities of more than 90% of the original material forming the microspheres. It is also obvious that the particles are spherical.

For further size analysis 5 g of the spray dried microcapsules were insolubilised by heating for 55 minutes at a temperature of 176° C. in a hot air oven. The microspheres were sized using a Coulter Multisizer 2E (trade mark) and a TAII Sampling Stand fitted with a 200 μm orifice tube which found that the volume median diameter of the microspheres was 71 μm and the modal size was 61 μm This size distribution can be found in FIG. 3. The larger size measured by the Coulter Counter is due to swelling of the microsphere in an aqueous environment.

### EXAMPLE 2

100 ml of diafiltered aqueous 31% w/v HSA solution (again as a model or carrier) was spray dried on a Niro Mobile Minor spray dryer using the following conditions:

| | |
|---|---|
| Inlet Temperature | 80° C. |
| Outlet Temperature | 48° C. |
| Atomisation Pressure | 1.0 barg |
| Feed Rate | 13.3 g/min |
| Atomisation Type | Two fluid nozzle |

Photomicrographs of the soluble spray dried product can be found in FIG. 4. The microspheres are nearly all solid and smaller than the product from Example 1. The minority of microspheres that contain air have thick walls imparting a high mechanical strength.

### EXAMPLE 3

150 ml of 39% w/v trehalose solution (equivalent to 64 g of trehalose dihydrate (Sigma Aldrich Company Ltd, Poole, Dorset) dissolved in water up to a volume of 150 ml) was spray dried on a Niro Mobile Minor spray dryer using a NT2 rotary atomiser (Newland Design, Lancaster) at the following conditions:

| | |
|---|---|
| Inlet Temperature | 200° C. |
| Outlet Temperature | 108° C. |
| Feed Rate | 6 g/min |
| Rotational Speed | 13,500 rpm |

These process conditions gave a product yield of 81%. The product (Batch NT2TRE1) obtained on microscopic examination suspended in vegetable oil showed a bimodal size distribution of microspheres with more than 99% of population solid containing no entrapped air. The geometric size distribution was determined using a API Aerosizer fitted with an Aerodispenser (Amherst Process Instruments Inc, Hadley, Mass.) using a high shear force, medium feed rate and a particle density of 1.56 g/cm³. The results from this anlysis showed that the main larger peak of the distribution had a modal size of 56 μm with the smaller fraction having a modal size of 28 μm. The size distribution obtained from the Aerosizer is shown in FIG. 5.

### EXAMPLE 4

Example 3 was repeated with the same feed concentration using higher rotational speeds for the NT2 atomiser at 16,400 rpm (Batch NT2TRE2) and 19,000 rpm (Batch NT2TRE3) with similar spray drying conditions. The subsequent microscopic and size analysis using the Aerosizer showed the following results (Table 1). The process yields were 94 and 89% respectively.

TABLE 1

| | | | Minor Peak | Major Peak |
|---|---|---|---|---|
| Batch | Atomiser | Percentage | Modal Size | Modal Size |
| Number | Speed (rpm) | Solid | (μm) | (μm) |
| NT2TRE2 | 16,400 | >99 | 22 | 47 |
| NT2TRE3 | 19,000 | >99 | 19 | 39 |

### EXAMPLE 5

The three products from Examples 3 and 4 were sieved to separate the two peaks of the bimodal distribution. 5 g of batch NT2TRE1 was placed in a 200 mm diameter stainless steel-test sieve (Endecotts, London) with an aperture size of 38 μm. The sieve was fitted with a lid and receiver and manually shaken for 5 minutes. The materials that were retained by and passed through the sieve were collected for assessment. Similarly 5 g of each of the products from batches NT2TRE2 and NT2TRE3 were sieved through 38 and 32 μm sieves respectively. The yield from the larger fraction retained by the sieve was in all cases greater than 60%. Microscopic examination showed a narrow size distribution and efficient separation of the two peaks of the bimodal size distribution. A photomicrograph of the fraction retained by the 32 μm sieve is shown in FIG. 6. The six fractions produced by sieving from the three batches were sized using the Aerosizer to give the results shown in Table 2.

TABLE 2

| | | Modal Size of | Modal Size of |
|---|---|---|---|
| | | Product retained | Product passed |
| | Sieve Aperture | by the Sieve | through the |
| Batch Number | Size (μm) | (μm) | Sieve (μm) |
| NT2TRE1 | 38 | 57 | 28 |
| NT2TRE2 | 38 | 47 | 22 |
| NT2TRE3 | 32 | 40 | 18 |

The Aerosizer size distributions are shown in FIG. 7 for the microspheres which passed through the sieves for batches NT2TRE3 and NT2TRE1 followed by the microspheres retained by the sieve for batches NT2TRE3, NT2TRE2 and NT2TRE1 in order of increasing size.

On further analysis of the geometric size distributions, the percentage of the particle population was calculated as shown in Table 3.

TABLE 3

| | | | | Percentage |
|---|---|---|---|---|
| | | | | of |
| | | | | Population |
| Modal Size | Lower Size | Upper Size | Size Range | within Size |
| (μm) | Limit (μm) | Limit (μm) | (μm) | Range |
| 18 | 16 | 26 | 10 | 70 |
| 28 | 24 | 36 | 12 | 70 |
| 40 | 37 | 53 | 16 | 70 |
| 47 | 43 | 61 | 18 | 70 |
| 57 | 52 | 72 | 20 | 70 |

The product that had a size of 40 μm also showed 75% of the particles were within a 17 μm size range and similarly 80% were within a 19 μm range.

### EXAMPLE 6

A feed solution was prepared by dissolving 7 g of trehalose octaacetate (Sigma Aldrich Company Ltd, Poole, Dorset) and 3 g of nifedipine (Seloc France, Limay) in acetone to a volume of 50 ml. The resulting solution had a total solids loading of 20% w/v. This feed solution was spray dried on a Niro Mobile Minor spray dryer using the NT2 rotary atomiser using the following conditions:

| | |
|---|---|
| Inlet Temperature | 65° C. |
| Outlet Temperature | 46° C. |
| Feed Rate | 10 g/min |
| Rotational Speed | 14,600 rpm |

A product yield of 78% was obtained from these process conditions. The product when assessed using optical microscopy showed a bimodal size distribution of solid microspheres with modal sizes of around 44 μm and 20 μm when compared to a reference graticule.

### EXAMPLE 7

100 ml of 14% w/v raffinose pentahydrate solution (14 g of raffinose pentahydrate (Pfanstiehl, Waukegan, Ill.) dissolved in water to a volume of 100 ml) was spray dried on a Niro Mobile Minor spray dryer using a NT2 rotary atomiser at the following conditions:

| | |
|---|---|
| Inlet Temperature | 170° C. |
| Outlet Temperature | 82° C. |
| Feed Rate | 10 g/min |
| Rotational Speed | 13,500 rpm |

The product obtained, with a process yield of 68%, showed on microscopic examination a bimodal size distribution of solid microspheres containing no entrapped air. The size distribution was determined on the Aerosizer using the same analytical conditions as Example 3 and a particle density of 1.47 g/cm³. The results from this analysis gave a main larger distribution with modal size of 36 μm with only a very small fraction having a modal size of 18 μm as shown in FIG. 8. On analysis of the distribution it was found that 70% of the microspheres were present within the 17 μm size range between 26 and 43 g. The raffinose pentahydrate is a carrier for a pharmacologically active compound.

### EXAMPLE 8

70 ml of a 31% w/v lidocaine solution in acetone (21.5 g of lidocaine (Sigma)) was spray dried on a Niro Mobile Minor spray dryer using a NT2 rotary atomiser at the following conditions:

| | |
|---|---|
| Inlet Temperature | 65° C. |
| Outlet Temperature | 45° C. |
| Feed Rate | 10 g/min |
| Rotational Speed | 13,500 rpm |

The product was spherical on optical assessment. The particle size distribution was bimodal with spherical solid microspheres having modal sizes of 41 μm and 20 μm.

### EXAMPLE 9

A solution was prepared by dissolving 38 g of trehalose dihydrate and 2 g diltizem hydrochloride (Lusochimica spa, Milan, Italy) in water to give a total volume of 100 ml. This solution was spray dried using the NT2 atomiser and Mobile Minor spray drier using the following conditions:

| | |
|---|---|
| Inlet Temperature | 200° C. |
| Outlet Temperature | 105° C. |
| Feed Rate | 11 g/min |
| Rotational Speed | 13,500 rpm |

A process yield of 94% was obtained. On microscopic examination, the smooth and spherical particles produced exhibited a bimodal size distribution with less than 2% of the particles containing small amounts of entrapped air. This was confirmed when sized using the Aerosizer, according to the conditions and density described in Example 3. This showed that the major peak which contained the larger microspheres had a modal size of 43 μm and the smaller peak had a mode of 20 μm. The geometric size distribution showed that 70% of the particle population was in the range of 36 to 56 μm which is a 20 μm size range.

### EXAMPLE 10

A solution was prepared by dissolving 38 g of trehalose dihydrate and 2 g of a model protein in the form of human serum albumin (Sigma) in water to give a total volume of 100 ml. This solution was spray dried as described in Example 9. In common with Example 9, similar process yields and particle characteristics were obtained. To evaluate whether the spray drying had either degraded or polymerised the albumin, gel electrophoresis under non-reducing conditions was carried out using reference lyophilised albumin and molecular markers. This showed that the albumin was unaffected by the spray drying process. This was also confirmed by gel permeation chromatography which demonstrated that no additional dimerisation or polymerisation had occurred.

1. Microparticles comprising or consisting of a therapeutic agent, having a relative particle density of at least 80% of the agent, and a shape factor of 1 to 5.

2. Microparticles according to claim 1, obtainable by spray-drying from solution or suspension.

3. Microparticles according to claim 1 or claim 2, wherein the relative particle density is at least 90%.

4. Microparticles according to any preceding claim, wherein at least 95% of the particles by weight have a diameter of 10-500 μm.

5. Microparticles according to claim 4, wherein at least 95% of the particles have a diameter of 20-200 μm.

6. Microparticles according to claim 4, wherein at least 95% of the particles have a diameter of 30-100 μm.

7. Microparticles according to any preceding claim, wherein the shape factor is 1 to 2.

8. Microparticles according to any preceding claim, consisting of the therapeutic agent and, optionally, an excipient.

9. Use of microparticles according to any preceding claim for the manufacture of a medicament for administration by needleless injection.

10. A needleless syringe comprising microparticles according to any of claims 1 to 8.

11. A method of therapeutic treatment which comprises the transdermal, transmucosal or subcutaneous delivery of microparticles according to any of claims 1 to 8 using a needleless syringe.

12. A method of producing microparticles according to any one of claims 1 to 8 which comprises spray-drying a solution or a suspension comprising the therapeutic agent.

13. A method as claimed in claim 12, wherein the spray drying comprises the use of a rotary atomiser.
